(19) United States
(12) Patent Application Publication
(10) Pub. No.: US 2009/0105671 A1
(43) Pub. Date: Apr. 23, 2009

(54) FIBROUS DRESSING

(76) Inventors: Anthony C. Daggar, North Yorkshire (GB); Helen Floyd, North Yorkshire (GB)

Correspondence Address: KNOBBE MARTENS OLSON & BEAR LLP, 2040 MAIN STREET, FOURTEENTH FLOOR, IRVINE, CA, 92614, US

(21) Appl. No.: 12/094,963

(22) PCT Filed: Nov. 27, 2006
(86) PCT No.: PCT/GB2006/004379

§ 371 Date: Sep. 23, 2008

(30) Foreign Application Priority Data
Nov. 25, 2005 (GB) 0524027.0

(51) Int. Cl. A61F 13/02; A61M 1/00; B29C 47/00

(52) U.S. Cl. 604/305; 604/313; 604/290; 264/465

(57) ABSTRACT

An apparatus for aspirating and/or cleansing wounds comprising a flow path comprising a wound dressing having a backing layer and at least one outlet pipe for connection to a fluid off take tube and which has a wound contact integer which in use is in contact with the wound bed and in which the wound contact integer comprises a biodegradable and/or bioabsorbable fibrous electrospun scaffold.

The present invention relates to a medical wound dressing for aspirating, irrigating and/or cleansing wounds, and a method of treating wounds using such apparatus for aspirating, irrigating and/or cleansing wounds.

It relates in particular to such a wound dressing and method that can be easily applied to a wide variety of wounds, but in particular chronic, wounds, to cleanse them of materials that are deleterious to wound healing, whilst distributing within the wound or returning to the wound materials that are beneficial in some therapeutic aspect, in particular to wound healing.

It is known to apply negative pressure therapy to a range of dermal wounds, including acute, sub acute, chronic, traumatic and dehisced wounds. The therapy is used to remove wound exudate, reduce the bacterial burden and facilitate granulation tissue formation, and thus to facilitate the healing of the wound. In known forms of such wound therapy, only aspiration of the wound, the sequential aspiration and irrigation of the wound, or the irrigation and aspiration of the wound, in combination with cleansing and re-circulation of exudates back to the wound bed after the removal of detrimental factors, may be carried out.

Many such forms of negative pressure wound therapy tend to use a specific dressing, which has a fibrous, mesh or perforated integer, such as a reticulated sponge that is drawn onto the wound bed by the sub-atmospheric pressure, and is said to prevent overgrowth of the granulation tissue that is stimulated by the negative pressure. However, pain may be associated with negative pressure therapy. This pain can be due to the in-growth of granulation tissue into the sponge dressing or integer after a short period of time, and after such in-growth of the wound bed tissue, dressing removal can increase pain further.

It is also known, in a method of treating wounds with negative pressure therapy, to apply a scaffold to the wound bed (between the wound and the wound dressing contact layer) to create a favourable environment for tissue repair. Such scaffolds may be bio-scaffolds or synthetic scaffolds, may be biodegradable and/or bioabsorbable (and can remain within a wound), or non-biodegradable and/or non-bioabsorbable (and need to be removed.)

Such a scaffold is separate from the dressing which it underlies, so that such forms of negative pressure wound therapy require multiple application of different integers over the wound bed. For the same reason, the whole assembly tends to be less than optimally conformable. The scaffolds undergo infiltration by granulation tissue and cells, and nonbio-absorbable/-degradable scaffolds, used in known negative pressure therapy, suffer from the disadvantages of needing to be removed during or after wound healing, and on removal they cause damage to the wound bed and pain to the patient.

It would be desirable to provide a dressing which avoids these disadvantages of known negative pressure therapy scaffolds and can be easily applied to a wide variety of wounds, but in particular chronic, wounds, in an apparatus for aspirating, irrigating and/or cleansing wounds.

We have surprisingly found that this may be achieved by an electrospun scaffold that is an integral part of a negative pressure therapy dressing, e.g. of the underside of a wound contact layer in such a dressing.

Accordingly, in a first aspect of the present invention there is provided a conformable wound dressing that comprises a backing layer with a wound-facing face which is capable of forming a relatively liquid-tight seal or closure over a wound, and has at least one outlet pipe for connection to a fluid offtake tube, which passes through and/or under the wound-facing face, the point at which the or each outlet pipe passes through and/or under the wound-facing face forming a relatively fluid-tight seal or closure over the wound, and a wound contact integer which in use lies in contact with the wound bed, characterised in that the wound contact integer comprises a biodegradable and/or bioabsorbable fibrous electrospun scaffold which in use lies in contact with the wound bed.

According to the present invention there is provided an apparatus for aspirating, and/or cleansing wounds comprising
(a) a flow path comprising a wound dressing having a backing layer with a wound facing face and at least one outlet pipe for connection to a fluid offtake tube, which passes through and/or under the wound-facing face;
(b) at least one device for moving fluid through the wound dressing and a wound contact integer which in use lies in contact with the wound bed;
(c) characterised in that the wound contact integer comprises a biodegradable and/or bioabsorbable fibrous electrospun scaffold.

In one embodiment of the first aspect of the invention, the fibrous electrospun scaffold which in use lies in contact with the wound bed comprises nanofibres that are electrospun directly onto and adhere to the wound contact integer as the electrospinning target.

In an additional embodiment of the first aspect of the invention, the fibrous electrospun scaffold which in use lies in contact with the wound bed comprises nanofibres that are electrospun directly onto the assembled dressing.

When used herein the term ‘nanofibres’ means the majority of fibre diameters in the scaffold are less than 1 m.

The dressing of the first aspect of the invention avoids the disadvantages of known negative pressure therapy scaffolds. It can be easily applied to a wide variety of wounds, but in particular chronic wounds, in an apparatus for aspirating, irrigating and/or cleansing wounds. It has the advantage of being a single application product. It is also more conformable as it is attached to the rest of the dressing.

In use, the scaffold is gradually released from the wound contact integer of the dressing by wound exudate, the action of which may be enhanced by irrigation of the dressing, so that none of the scaffold requires removal from the wound bed on dressing removal. This results in reduced pain and discomfort experienced by the patient and the reduction in the need to use topical anaesthetic.

The electrospun material of the scaffold tends to be in the form of nanofibres, leading to a higher surface area, higher porosity and increased fluid movement due to the high porosity, and hence faster degradation, compared to a standard bioscaffold.

In one embodiment of the first aspect of the invention, the dressing has no further pipe or pipes that pass through and/or under the wound-facing face and communicate with the wound space under the wound-facing face. Such an embodiment is typically used for the aspiration of the wound, or the sequential aspiration and irrigation of the wound with flow reversal, through the outlet pipe or pipes.

During aspiration of the wound, optionally in the sequential aspiration and irrigation of the wound, a source of sub-atmospheric pressure is applied to the outlet pipe via connection to a fluid offtake tube. This draws the wound contact integer onto the wound bed, and it lies in contact with the wound bed until atmospheric pressure is restored.

In some embodiments of the present invention the wound area may be subject to a sub atmospheric pressure or negative pressure. This negative pressure may be between about 1.01 and 100.3 kPa (0.01 and 0.99 atmospheres). Other suitable pressure ranges include (but not limited to) between 60 and 80 mmHg (0.079 and 0.105 atmospheres, 9.12 and 10.7 kPa).

For shallower wounds, the wound contact integer which in use lies in contact with the wound bed may typically be the backing layer with a wound-facing face bearing the biodegradable and/or bioabsorbable fibrous electrospun scaffold which in use lies in contact with the wound bed.

Alternatively, for shallower wounds, the wound contact integer which in use lies in contact with the wound bed may typically be a wound filler as a component of a wound dressing under the backing layer with a wound-facing face bearing the biodegradable and/or bioabsorbable fibrous electrospun scaffold, which in use lies in contact with the wound bed.

Alternatively, for shallower wounds, the wound contact integer which in use lies in contact with the wound bed may typically be a wound filler as a component of a wound dressing under the backing layer which comprises or consist essentially of one or more conformable hollow bodies defining a manifold that
a) collects the fluid directly from the wound in aspiration or
b) also functions as an inlet pipe manifold that delivers fluid directly to the wound bed in irrigation.

It will have a wound-facing face bearing the biodegradable and/or more conformable hollow bodies defining bioabsorbable fibrous electrospun scaffold which in use lies in contact with the wound bed.

A more suitable wound contact integer for deeper wounds when the therapy is applied in this way may be one which comprises one or more conformable hollow bodies that at least partly surround(s) a solid integer. These may be defined by, for example, a polymer film, sheet or membrane. This may provide a system with better rigidity for convenient handling.

It will have a wound-facing face bearing the biodegradable and/or bioabsorbable fibrous electrospun scaffold which in use lies in contact with the wound bed.

Alternatively, in this form, the one or more conformable hollow bodies may define an outlet pipe manifold that
a) collects the fluid directly from the wound in aspiration or
b) also functions as an inlet pipe manifold that delivers fluid directly to the wound bed in irrigation.

It will have a wound-facing face bearing the biodegradable and/or more conformable hollow bodies defining bioabsorbable fibrous electrospun scaffold which in use lies in contact with the wound bed.

In another embodiment of the first aspect of the invention, the dressing has further pipe or pipes that pass through and/or under the wound-facing face and communicate with the wound space under the wound-facing face.

Such an embodiment is typically used for the simultaneous or sequential aspiration and irrigation of the wound without flow reversal, optionally in combination with cleansing and re-circulation of exudate back to the wound bed after the removal of detrimental factors.

The further pipe or pipes that pass through and/or under the wound-facing face and communicate with the wound space under the wound-facing face then function typically as at least one inlet pipe for connection to a fluid supply tube, which passes through and/or under the wound-facing face, for the supply of irrigant to the wound bed.

Accordingly, in one embodiment of the first aspect of the present invention there is provided a conformable wound dressing that comprises a backing layer with a wound-facing face which is capable of forming a relatively liquid-tight seal or closure over a wound, and has at least one inlet pipe for connection to a fluid supply tube, which passes through and/or under the wound-facing face, and
at least one outlet pipe for connection to a fluid offtake tube, which passes through and/or under the wound-facing face,
the point at which the or each inlet pipe and the or each outlet pipe passes through and/or under the wound-facing face forming a relatively fluid-tight seal or closure over the wound;
a wound contact integer which in use lies in contact with the wound bed, characterised in that
the wound contact integer comprises a biodegradable and/or bioabsorbable fibrous electrospun scaffold which in use lies in contact with the wound bed.

Again, for shallower wounds, the wound contact integer which in use lies in contact with the wound bed may typically be the backing layer with a wound-facing face bearing the biodegradable and/or bioabsorbable fibrous electrospun scaffold which in use lies in contact with the wound bed.

Alternatively, for shallower wounds, the wound contact integer which in use lies in contact with the wound bed may typically be a wound filler as a component of a wound dressing under the backing layer with a wound-facing face bearing the biodegradable and/or bioabsorbable fibrous electrospun scaffold which in use lies in contact with the wound bed.

Alternatively, for shallower wounds, the wound contact integer which in use lies in contact with the wound bed may typically be a wound filler as a component of a wound dressing under the backing layer which comprises or consist essentially of one or more conformable hollow bodies defining
a) an outlet pipe manifold that collects the fluid directly from the wound in aspiration or
b) more often an inlet pipe manifold that delivers fluid directly to the wound bed in irrigation.

It will have a wound-facing face bearing the biodegradable and/or more conformable hollow bodies defining bioabsorbable fibrous electrospun scaffold which in use lies in contact with the wound bed.

A more suitable wound contact integer for deeper wounds when the therapy is applied in this way may be one which comprises one or more conformable hollow bodies that at least partly surround(s) a solid integer. These may be defined by, for example, a polymer film, sheet or membrane,

This may provide a system with better rigidity for convenient handling. It will have a wound-facing face bearing the biodegradable and/or bioabsorbable fibrous electrospun scaffold which in use lies in contact with the wound bed.

Alternatively, in this form, the one or more conformable hollow bodies may define
a) an outlet pipe manifold that collects the fluid directly from the wound in aspiration or
b) more often an inlet pipe manifold that delivers fluid directly to the wound bed in irrigation.

It will have a wound-facing face bearing the biodegradable and/or more conformable hollow bodies defining bioabsorbable fibrous electrospun scaffold which in use lies in contact with the wound bed.

Examples of suitable biodegradable and/or bioabsorbable fibrous electrospun scaffolds include non-woven mats, pads, rolls and wadding of electrospun fibres, electrospun fibres into or onto woven materials or any form of support, or lyophilised, or in a gel, or sandwiched between any other substance e.g. alginate.

In some embodiments of the present invention the wound contact integer may also comprise gauze, foam or other porous material onto which and/or in which the biodegradable and/or bioabsorbable fibrous electrospun scaffold/material is situated.

The apparatus of the present invention may also have in addition, in some embodiments, gauze or foam or other porous material as a filler material under the backing layer of the wound dressing.

Examples of suitable biodegradable and/or bioabsorbable electrospun fibres include electrospun fibres of diameters in the range of less than 100 nm up to and above 100 μm, in continuous or truncated fibres.

Suitable electrospun fibre diameters may be in the range of 150 μm to 90 nm, 50 μm to 50 nm, 75 μm to 75 nm, 90 μm to 90 nm, 80 μm to 90 nm, 110 μm to 90 nm or 120 μm to 105 nm.

Examples of suitable depths of the scaffold are in the range from below 50 μm to above 3 mm.

Suitable depths of the scaffold may also include 25 μm to 3.1 mm, 40 μm to 3.1 mm, 45 μm to 3.25 mm, 49 μm to 3.1 mm or 50 μm to 2.5 mm.

Examples of suitable biodegradable and/or bioabsorbable materials to be deposited onto and/or attached to the surface of the dressing wound contact integer include biodegradable and/or bioabsorbable materials such as

polylactic acid, polyglycolic acid, poly(D-)lactic acid, polyglycoliclactic acid, keratin, laminin, elastin, collagen and naturally occurring extracellular matrix proteins,
polydioxanones, polycaprolactone, and blends and co-polymers thereof.

The materials may be electrospun to be deposited onto and/or attached to the surface of the dressing wound contact integer by any means known to those skilled in the art.

Preferably, they are solvent spun using appropriate solvents, such as dimethylformamide, methylene chloride, chloroform, dichloromethane, acetonitrile, methanol, N-methylpyrolidone, hexafluoroisopropanol and dimethyl sulphoxide, with appropriate additives, such as sodium chloride, magnesium chloride, potassium dihydrogen phosphate, potassium iodate and potassium phosphate calcium carbonate, calcium phosphate and calcium lactate, in solution form or in nanoparticulate forms, and any other additives, solvents, polymers, bioactives, pharmaceutical agents, metals, metal oxides or cells or cellular components known to one skilled in the art, that can be integrated into an electrospun format.

According to the present invention there is provided a method of manufacturing a wound dressing comprising of the steps of: electrospinning, a biodegradable and/or bioabsorbable fibrous material onto a wound contact integer of a wound dressing which in use the wound contact integer lies in contact with the wound bed.

According to the present invention there is provided a method of treating wounds to promote wound healing using the apparatus according to claim 1 in which a negative pressure is applied to the wound.

The present invention is illustrated by the following figures and examples:

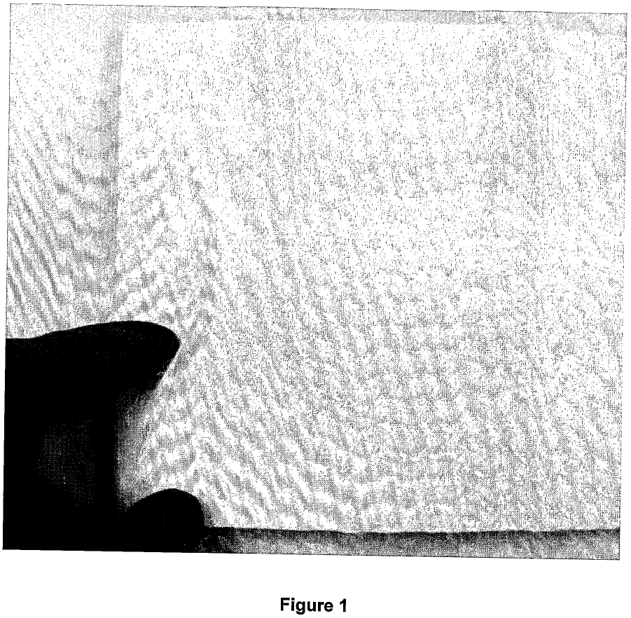

FIG. 1 Shows an electrospun polycaprolactone fibre mat partially peeled from ALLEVYN™ Non-Adhesive dressing.

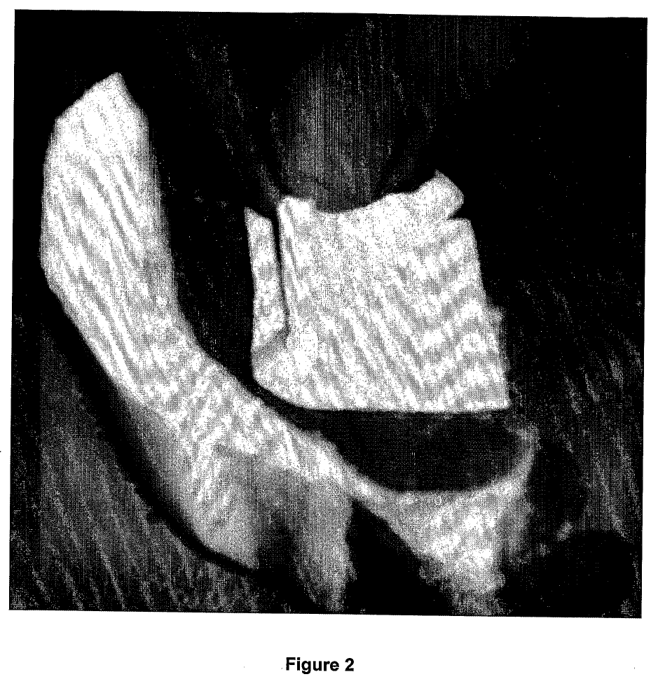

FIG. 2 Shows removal of ALLEVYN™ Non-Adhesive dressing from an electrospun polycaprolactone fibre mat, which has adhered to the surface of pork after 6 hours 45 minutes under vacuum.

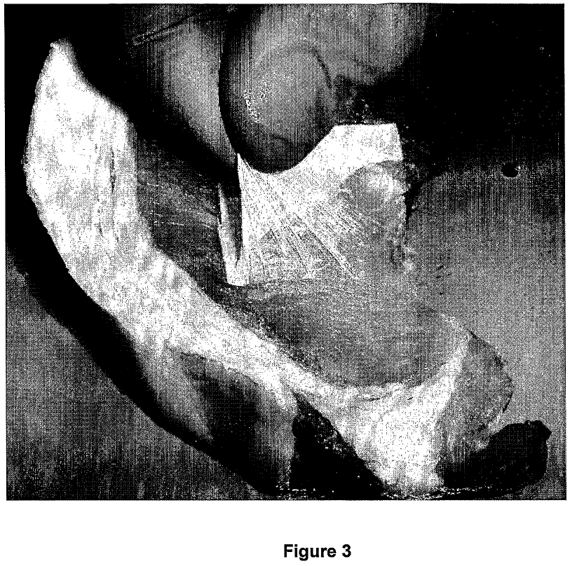

FIG. 3 Shows removal of an electrospun polycaprolactone fibre mat from the surface of pork after 6 hours 45 minutes under vacuum.

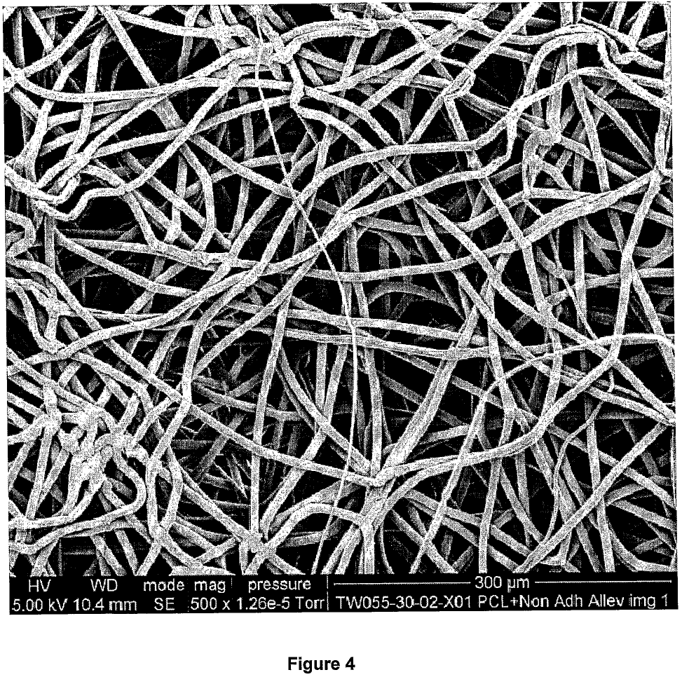

FIG. 4 Shows scanning electron micrograph of an electrospun polycaprolactone fibre mat deposited on ALLEVYN™ Non-Adhesive dressing.

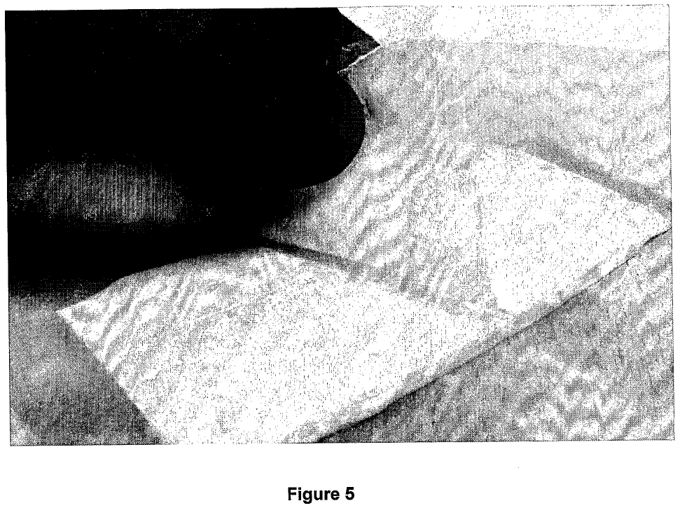

FIG. 5 Shows electrospun polycaprolactone fibre mat partially peeled from wet ALLEVYN™ Adhesive dressing.

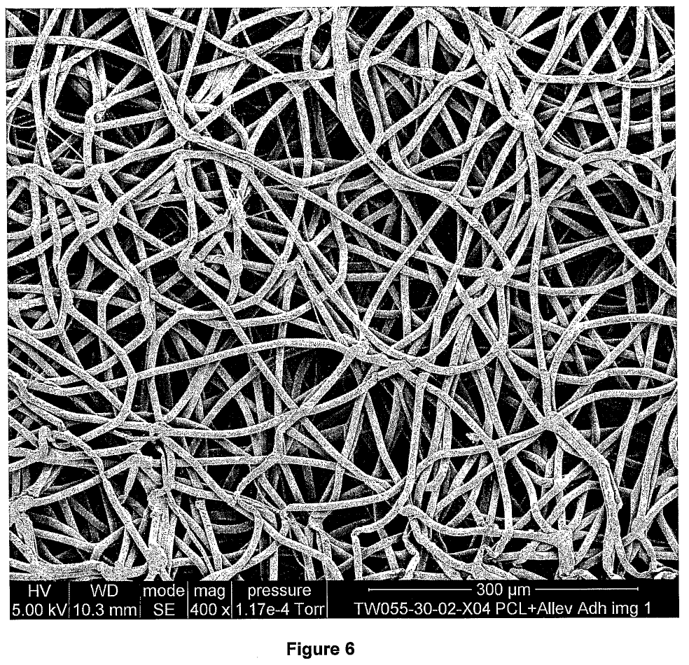

FIG. 6 Shows scanning electron micrograph of an electrospun polycaprolactone fibre mat deposited on ALLEVYN™ Adhesive dressing.

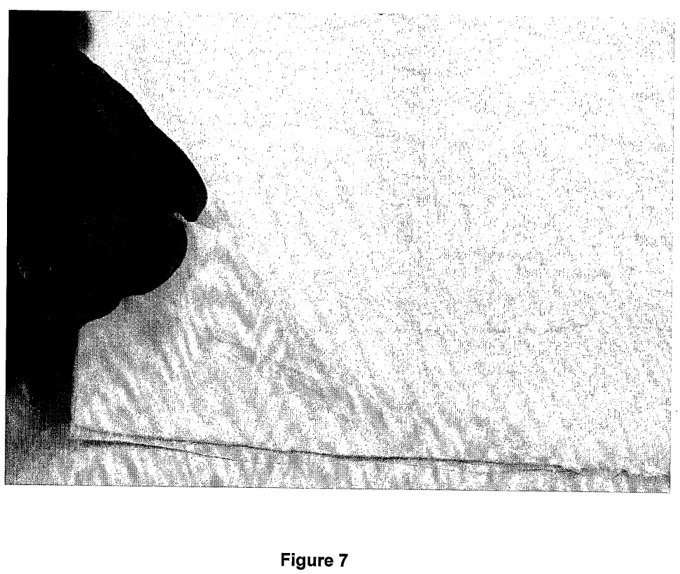

FIG. 7 Shows electrospun polycaprolactone fibre mat partially peeled from polyurethane foam.

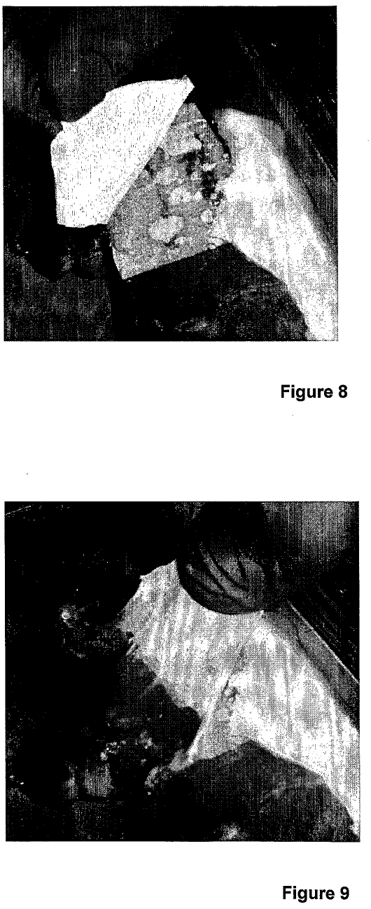

FIG. 8 Shows removal of polyurethane foam from an electrospun polycaprolactone fibre mat, which has adhered to the surface of pork after 6 hours 45 minutes under vacuum.

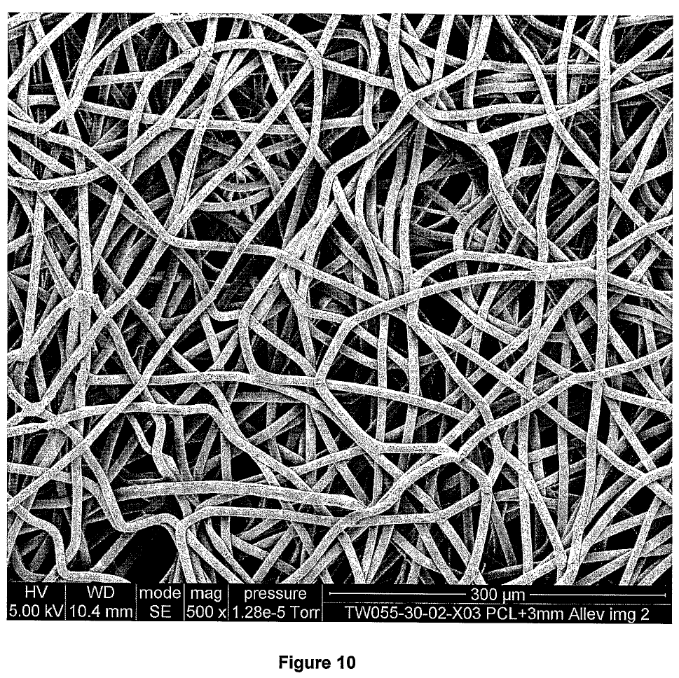

FIG. 9 Shows removal of an electrospun polycaprolactone fibre mat from the surface of pork after 6 hours 45 minutes under vacuum.

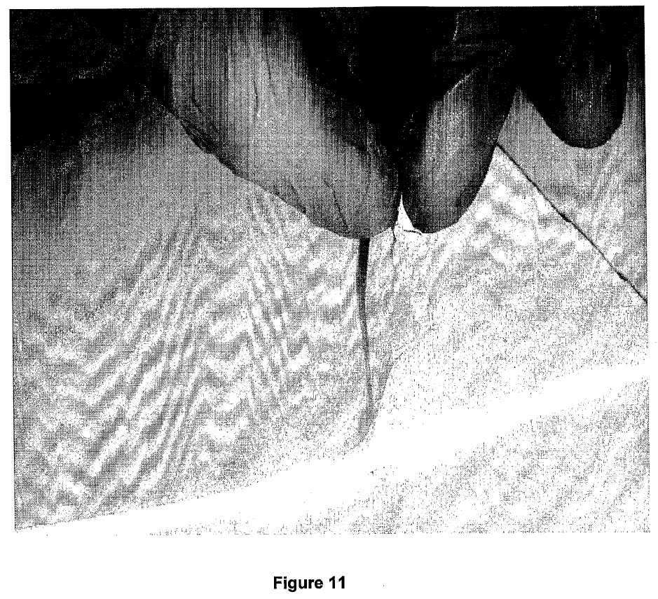

FIG. 10 Shows a scanning electron micrograph of an electrospun polycaprolactone fibre mat deposited on polyurethane foam.

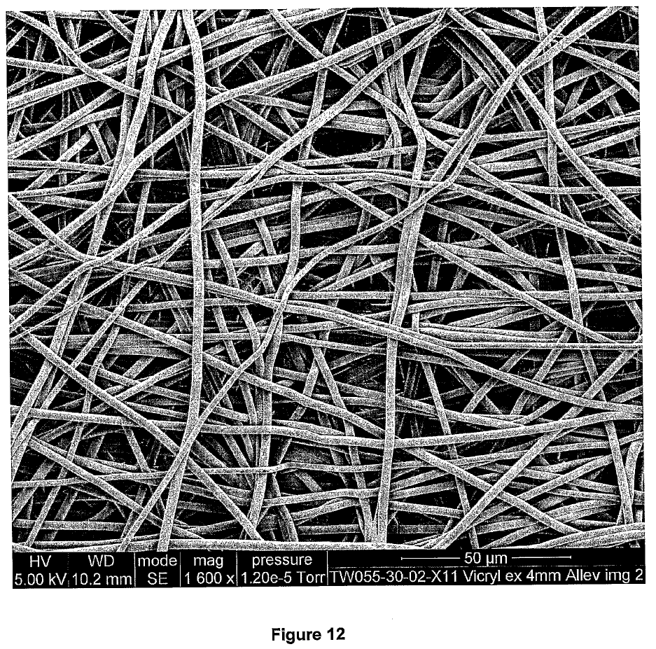

FIG. 11 Shows electrospun poly(lactic acid/glycolic acid) copolymer fibre mat partially peeled from polyurethane foam.

FIG. 12 Shows a scanning electron micrograph of an electrospun poly(lactic acid/glycolic acid) copolymer fibre mat deposited on polyurethane foam.

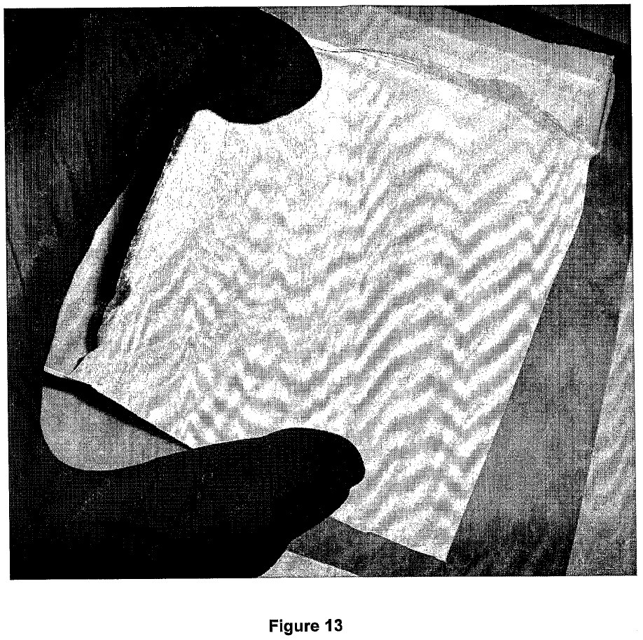

FIG. 13 Shows electrospun poly(lactic acid/glycolic acid) copolymer fibre mat partially peeled from poly(ethylene/vinyl acetate)/polystyrene film.

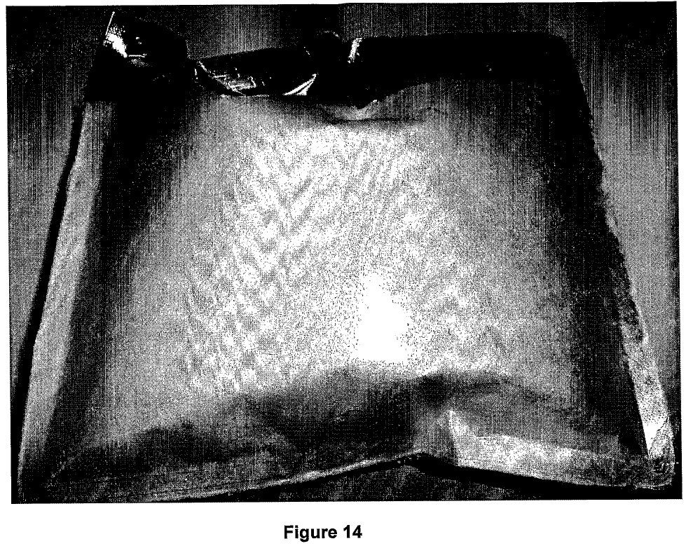

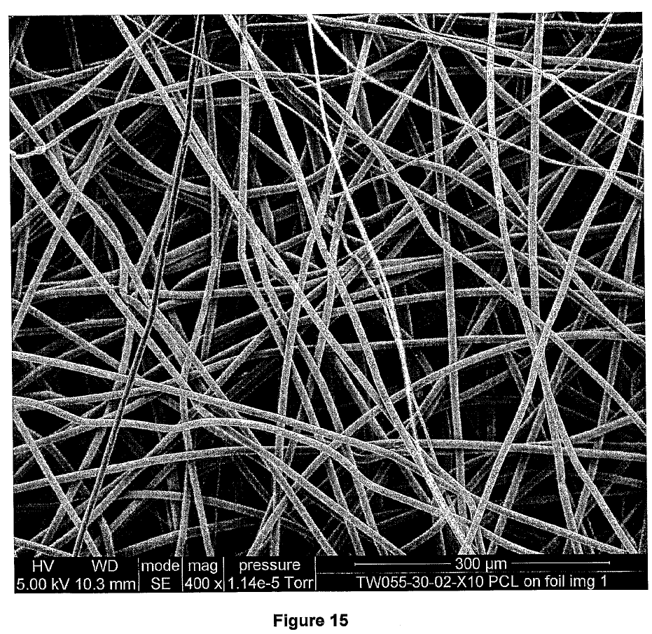

FIG. 14 Shows an inflated foil/polymer laminate hollow body with a layer of electrospun polycaprolactone fibres.

FIG. 15 Shows scanning electron micrograph of the electrospun polycaprolactone fibre mat deposited on a foil/laminate hollow body.

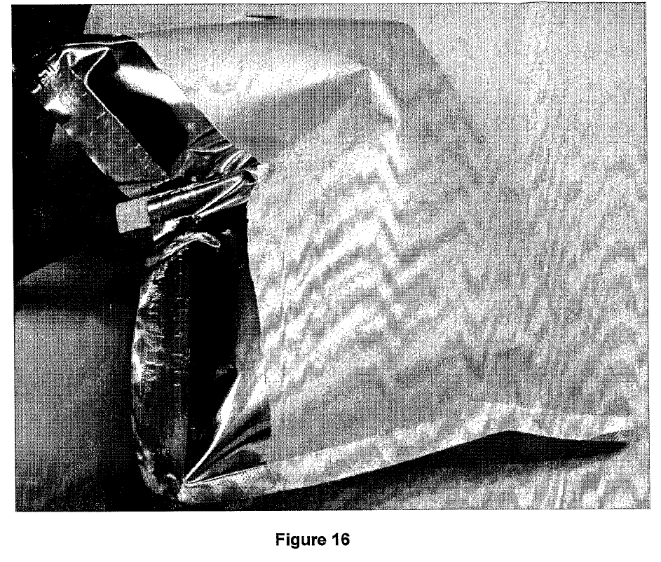

FIG. 16 Shows an inflated foil/polymer laminate hollow body with a layer of electrospun poly(lactic acid/glycolic acid) copolymer fibres.

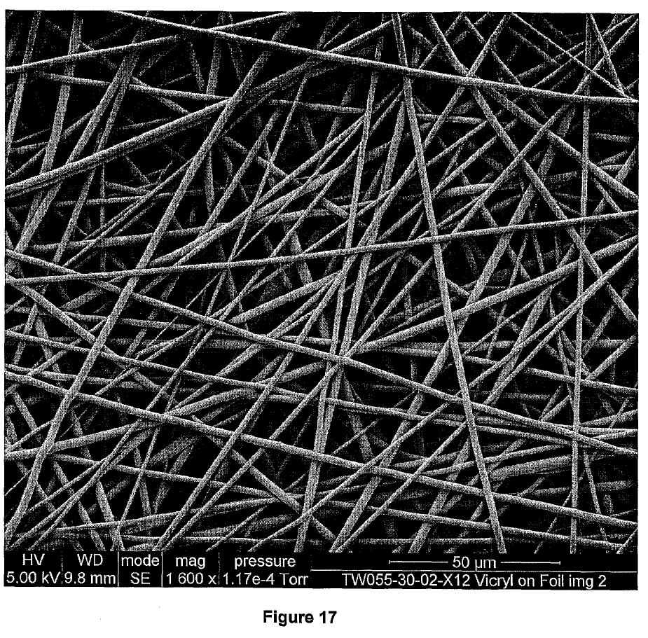

FIG. 17 Shows a scanning electron micrograph of an electrospun poly(lactic acid/glycolic acid) copolymer fibre mat deposited on a foil/laminate hollow body.

### EXAMPLE 1

### Fibre Mat Electrospun Onto a Polyurethane Dressing Wound Contact Layer

A 10% solution of polylacticglycolic acid (25% lactide, 75% glycolide) copolymer was prepared in dichloromethane. This was electrospun by passing through a needle at a flow rate of 0.03 ml/min and applying a DC electric potential of 20 kV with a working distance of 15 cm onto the wound contact layer of an ALLEVYN™ non-adhesive dressing (Smith & Nephew) placed on a grounded stainless steel target. A thin mat of electrospun material was deposited onto the wound contact layer within about 10 minutes and this was allowed to air dry. The electrospun mat was found to be well attached to the surface of the wound contact layer and was quite conformable.

### EXAMPLE 2

### Fibre Mat Electrospun Onto a Polyurethane Dressing

A 20% solution of polycaprolactone homopolymer was prepared in dichloromethane. This was electrospun by passing through a needle at a flow rate of 0.03 mLmin⁻¹ and applying a DC electric potential of 6.5-25 kV with a working distance of 15 cm onto the wound contact layer of an ALLEVYN™ Non-Adhesive polyurethane dressing (Smith & Nephew) placed onto a rotating grounded stainless steel mandrel. A 150-200 μm thick mat of electrospun material comprising fibres 6.5-8.7 μm in diameter was deposited onto the wound contact layer within 136 minutes. The electrospun fibre mat was found to be well attached to the surface of the wound contact layer, but could be peeled off intact upon application of direct force.

After the ALLEVYN™ dressing incorporating the layer of electrospun fibres had been applied to the surface of raw pork and dried in a vacuum oven for at least 3 hours at room temperature and a pressure of less than or equal to 10 mbar, the ALLEVYN™ dressing could be easily peeled from the electrospun fibre layer, which remained intact on the surface of the pork. Control pieces of ALLEVYN™ dressing without a layer of electrospun fibres adhered to the surface of the pork under identical conditions.

### EXAMPLE 3

### Fibre Mat Electrospun Onto a Polyurethane Dressing

A 20% solution of polycaprolactone homopolymer was prepared in dichloromethane. This was electrospun by passing through a needle at a flow rate of 0.03 mLmin⁻¹ and applying a DC electric potential of 7.0 kV with a working distance of 15 cm onto the wound contact layer of an ALLEVYN™ Adhesive polyurethane dressing (Smith & Nephew) placed onto a rotating grounded stainless steel mandrel. A mat of electrospun material comprising fibres 6.7-9.1 μm in diameter was deposited onto the wound contact layer within 70 minutes. The electrospun fibre mat was found to be securely attached to the surface of the wound contact layer, but could be peeled off intact upon application of direct force after wetting the surface of the dressing. The peeled electrospun fibre mat was 70-90 μm thick.

### EXAMPLE 4

### Fibre Mat Electrospun Onto a Polyurethane Foam

A 20% solution of polycaprolactone homopolymer was prepared in dichloromethane. This was electrospun by passing through a needle at a flow rate of 0.03 mLmin⁻¹ and applying a DC electric potential of 7.0 kV with a working distance of 15 cm onto the 3 mm thick polyurethane foam component of an ALLEVYN™ polyurethane dressing (Smith & Nephew) without the wound contact layer or backing layer, which was placed onto a rotating grounded stainless steel mandrel. A 70-100 μm thick mat of electrospun material comprising fibres 7.4-9.3 μm in diameter was deposited onto the foam surface within 60 minutes. The electrospun fibre mat was found to be well attached to the surface of the foam, but could be peeled off intact upon application of direct force.

After the polyurethane foam incorporating the layer of electrospun fibres had been applied to the surface of raw pork and dried in a vacuum oven for at least 3 hours at room temperature and a pressure of less than or equal to 10 mbar, the polyurethane foam could be easily peeled from the electrospun fibre layer, which remained intact on the surface of the pork. Control pieces of ALLEVYN™ dressing without a layer of electrospun fibres adhered to the surface of the pork under identical conditions.

### EXAMPLE 5

### Fibre Mat Electrospun Onto a Polyurethane Foam

A 10% solution of poly(lactic acid/glycolic acid) (10% lactide, 90% glycolide) copolymer was prepared in hexafluoroisopropanol. This was electrospun by passing through a needle at a flow rate of 0.03 mLmin⁻¹ and applying a DC electric potential of 5.0 kV with a working distance of 15 cm onto the 4 mm thick polyurethane foam component of an ALLEVYN™ polyurethane dressing (Smith & Nephew) without the wound contact layer or backing layer, which was placed onto a rotating grounded stainless steel mandrel. A 60-80 μm thick mat of electrospun material comprising fibres 1.7-2.7 μm in diameter was deposited onto the foam surface within 120 minutes. The electrospun fibre mat was found to be well attached to the surface of the foam, but could be peeled off intact upon application of direct force.

### EXAMPLE 6

### Fibre Mat Electrospun Onto a Poly(ethylene/vinyl acetate)/polystyrene Film

A 10% solution of poly(lactic acid/glycolic acid) (10% lactide, 90% glycolide) copolymer was prepared in hexafluoroisopropanol. This was electrospun by passing through a needle at a flow rate of 0.03 mLmin⁻¹ and applying a DC electric potential of 7.0 kV with a working distance of 15 cm onto a poly(ethylene/vinyl acetate)/polystyrene film placed onto a rotating grounded stainless steel mandrel. The film comprised a non-perforated poly(ethylene/vinyl acetate)/polystyrene film used in the manufacture of the wound contact layer of ALLEVYN™ Cavity (Smith & Nephew). A 70-90 μm thick mat of electrospun material was deposited onto the polymer film within 120 minutes. The electrospun fibre mat was found to be well attached to the surface of the polymer film, but could be peeled off intact upon application of direct force.

### EXAMPLE 7

### Fibre Mat Electrospun onto a Hollow Body Formed from a Foil/polymer Laminate

A 20% solution of polycaprolactone homopolymer was prepared in dichloromethane. This was electrospun by passing through a needle at a flow rate of 0.03 mLmin⁻¹ and applying a DC electric potential of 7.0 kV with a working distance of 15 cm onto the polypropylene layer of a foil/polymer laminate hollow body placed onto a rotating grounded stainless steel mandrel. The hollow body was prepared by heat sealing together two sheets of four-layer laminate comprising oriented polypropylene, polyethylene, aluminium foil and polyethylene (FR 2175-B, Covalence Coated Products) so that the polypropylene layer formed the external layer. A 130-240 μm thick mat of electrospun material comprising fibres 7.0-9.7 pm in diameter was deposited onto the hollow body within 90 minutes. The electrospun fibre mat was found to be well attached to the surface of the hollow body, but could be peeled off intact upon application of direct force. The electrospun fibre mat did not detach from the hollow body after several cycles of inflation and deflation.

### EXAMPLE 8

### Fibre Mat Electrospun Onto a Hollow Body Formed from a Foil/polymer Laminate

A 10% solution of poly(lactic acid/glycolic acid) (10% lactide, 90% glycolide) copolymer was prepared in hexafluoroisopropanol. This was electrospun by passing through a needle at a flow rate of 0.03 mLmin⁻¹ and applying a DC electric potential of 5.0-7.0 kV with a working distance of 15 cm onto a foil/polymer laminate hollow body placed onto a rotating grounded stainless steel mandrel. The hollow body was prepared by heat sealing together two sheets of four-layer laminate comprising oriented polypropylene, polyethylene, aluminium foil and polyethylene (FR 2175-B, Covalence Coated Products) so that the polypropylene layer formed the external layer. A 30-90 μm thick mat of electrospun material comprising fibres 1.2-2.4 μm in diameter was deposited onto the hollow body within 90 minutes. The electrospun fibre mat was found to be well attached to the surface of the hollow body, but could be peeled off intact upon application of direct force. The electrospun fibre mat did not detach from the hollow body after several cycles of inflation and deflation.

1-21. (canceled)

22. An apparatus for use in treating a wound comprising:
a wound dressing having a backing layer with a wound-facing face and at least one outlet pipe for fluid offtake; and
a wound contact integer which is configured to lie in contact with the wound;
wherein the wound contact integer comprises a scaffold comprising electrospun fibres, the fibres being at least one of biodegradable and bioabsorbable.

23. The apparatus of claim 22, wherein the outlet pipe passes through the wound-facing face.

24. The apparatus of claim 22, wherein the outlet pipe passes under the wound-facing face.

25. The apparatus of claim 22, wherein the outlet pipe passes through and under the wound-facing face.

26. The apparatus of claim 22, further comprising at least one device for moving fluid through the wound dressing.

27. The apparatus of claim 22, wherein the backing layer is capable of forming a relatively fluid tight seal or closure over the wound.

28. The apparatus of claim 22, further comprising an inlet pipe for fluid irrigation.

29. The apparatus of claim 28, wherein the apparatus is configured to permit sequential aspiration and irrigation of the wound.

30. The apparatus of claim 22, wherein the apparatus is configured to form a relatively fluid-tight seal over the wound and between the outlet pipe and the wound dressing.

31. The apparatus of claim 22, wherein the electrospun fibres of the scaffold are electrospun directly onto the wound contact integer as the electrospinning target so as to adhere to the wound contact integer.

32. The apparatus of claim 22, wherein the electrospun fibres of the scaffold are electrospun directly onto the wound dressing.

33. The apparatus of claim 22, wherein the electrospun fibres of the scaffold comprise nanofibres.

34. The apparatus of claim 22, further comprising a source of sub-atmospheric or negative pressure applied to the wound.

35. The apparatus of claim 34, wherein the source of sub-atmospheric or negative pressure applied is between 1.01 kPa and 100.3 kPa.

36. The apparatus of claim 22, wherein the wound contact integer is the wound-facing face of the backing layer.

37. The apparatus of claim 22, wherein the wound contact integer is a wound filler situated under the backing layer.

38. The apparatus of claim 37, wherein the wound filler comprises of one or more hollow bodies defining a manifold.

39. The apparatus of claim 38, wherein the one or more hollow bodies defining a manifold has a wound-facing face bearing the scaffold of the wound contact integer.

40. The apparatus of claim 22, wherein the scaffold comprises a material selected from the group consisting of a non-woven mat, a pad of electrospun fibres, a roll of electrospun fibres, a wadding of electrospun fibers, a woven material, a lyophilized material, a gel and a sandwiched structure.

41. The apparatus of claim 22, wherein the scaffold comprises electrospun fibres into or onto woven material or any form of support.

42. The apparatus of claim 22, wherein the electrospun fibres include electrospun fibres of diameters in the range of between 100 nm and 100 μm, in continuous or truncated fibres.

43. The apparatus of claim 22, wherein the scaffold has a depth in the range between 50 μm and 3 mm.

44. A method of manufacturing a wound dressing comprising:
electrospinning fibrous material onto a wound contact integer of a wound dressing, the fibrous material being at least one of biodegradable and bioabsorbable, wherein the wound dressing is configured so that the wound contact integer lies in contact with a wound bed.

45. The method of claim 44, wherein the dressing comprises a backing layer with a wound-facing face, and the wound contact integer is the wound-facing face of the backing layer.

46. The method of claim 44, wherein the dressing comprises a backing layer with a wound-facing face, and the wound contact integer is a wound filler forming a component of the wound dressing situated under the backing layer.

47. A method of treating wounds to promote wound healing comprising:
providing a wound dressing apparatus comprising:
a wound dressing having a backing layer with a wound-facing face and at least one outlet pipe for fluid offtake; and
a wound contact integer comprising a scaffold comprising electrospun fibres, the fibres being at least one of biodegradable and bioabsorbable; and
positioning the wound dressing over a wound with the wound contact integer lying in contact with the wound.

48. The method of claim 47, further comprising applying a negative pressure to the wound.

49. The method of claim 48, wherein the negative pressure is between about 1.01 and 100.3 kPa.

50. The method of claim 48, wherein the negative pressure is between 60 and 80 mmHg.

51. The method of claim 47, further comprising removing the wound dressing from the wound with the scaffold comprising electrospun fibers remaining in contact with the wound.

52. The method of claim 47, further comprising aspirating fluid from the wound via the outlet pipe.

53. The method of claim 52, further comprising providing fluid irrigation to the wound.

54. The method of claim 47, wherein the wound contact integer is the wound-facing face of the backing layer.

55. The method of claim 47, wherein the wound contact integer is a wound filler situated under the backing layer.
